(19) United States
(12) Patent Application Publication
(10) Pub. No.: US 2022/0160670 A1
(43) Pub. Date: May 26, 2022

(54) COMPOSITION COMPRISING L-CARNITINE FOR THE TREATMENT OF MALE INFERTILITY

(71) Applicant: LABORATOIRE DES GRANIONS, Monaco (MC)

(72) Inventors: Amine Achite-Henni, Saint-Cloud (FR); Mohamed Benahmed, Nice (FR); Véronique Isnard, Nice (FR)

(73) Assignee: LABORATOIRE DES GRANIONS, Monaco (MC)

(21) Appl. No.: 16/770,024

(22) PCT Filed: Dec. 6, 2017
(86) PCT No.: PCT/EP2017/081707

§ 371 Date: May 14, 2021

(51) Int. Cl. A61K 31/205; A61K 31/202; A61K 31/122; A61K 31/355; A61K 31/4415; A61K 31/375; A61K 31/07; A61K 31/7004; A61K 31/519; A61K 31/714; A61K 31/593; A61K 31/01; A61K 31/198; A61K 33/04; A61K 33/30; A61K 47/12; A61K 47/26; A61K 35/60; A61K 31/145; A61K 36/33; A61K 36/752; A61K 36/82; A61K 36/15; A61K 36/63; A61K 36/28; A61K 36/05; A61K 36/185; A61P 15/08

(52) U.S. Cl. CPC A61K 31/205; A61P 15/08; A61K 31/122; A61K 31/355; A61K 31/4415; A61K 31/375; A61K 31/07; A61K 31/7004; A61K 31/519; A61K 31/714; A61K 31/593; A61K 31/01; A61K 31/198; A61K 33/04; A61K 33/30; A61K 47/12; A61K 47/26; A61K 35/60; A61K 31/145; A61K 36/33; A61K 36/752; A61K 36/82; A61K 36/15; A61K 36/63; A61K 36/28; A61K 36/05; A61K 36/185; A61K 31/202

(57) ABSTRACT

The invention relates to a composition for the treatment of male infertility. The invention is characterized by a composition comprising L-carnitine and/or acetyl-L-carnitine or prodrugs thereof for the treatment of infertility in men with a sperm concentration of less than 5 million spermatozoa per milliliter.

### FIELD OF THE INVENTION

The present invention relates to a composition comprising L-carnitine and/or acetyl-L-carnitine or prodrugs thereof for treating male infertility.

### PRIOR ART

In France, 5% to 10% of couples encounter difficulties in having a child. In 21% of these cases, male infertility appears to be the cause of this problem.

To observe normal male fertility, several conditions must be fulfilled, namely:
normal production of spermatozoa by the testicles (spermatogenesis), in terms of both quality and quantity;
good circulation of the spermatozoa within the male genital organs, which implies a complete absence of obstacle at the epididymes, the vas deferens and the urethra; and
adequate ejaculation.

Any factor that may interfere with one of these mechanisms may be responsible for hypofertility, of even infertility (sterility) in the man.

Analysis of the sperm is the most important examination in assessing male fertility. The spermogram measures with precision parameters such as the number of spermatozoa, the motility of the spermatozoa, the size and shape of the spermatozoa, the volume of sperm and the proportion of certain substances normally present. The results of the spermogram may vary according to stress, the duration of abstinence and the absorption of alcohol, medication or drugs.

In order to judge the quality of the sperm, the following data are commonly (World Health Organization Standards, entitled WHO Laboratory Manual for the Examination and Processing of Human Semen, fifth edition, 2010) taken into consideration from the ejaculate:
The volume of the sperm is normally greater than 1.5 ml:
   if the volume is greater than 6 ml, hyperspermia is spoken of;
   if the volume is below 1.5 ml, hypospermia is spoken of.
The concentration of the spermatozoa is normally greater than or equal to 15 million/ml:
   if the concentration is above 200 million/ml, polyspermia is spoken of;
   if the concentration is below 15 million/ml, oligospermia is spoken of; severe oligospermia is spoken of when the concentration is below 5 million/ml; and
   in the total absence of spermatozoa in the ejaculate, azoospermia is spoken of.

Moreover, in terms of the present application, if the concentration is below 0.5 million/ml, very severe oligospermia is spoken of.

Finally, normally, if the concentration is between 0.01 million/ml and 0.1 million/ml, cryptozoospermia is spoken of.
The total mobility of the spermatozoa is normally greater than or equal to 40%:
   if the mobility is below 40%, asthenospermia is spoken of.
The normal morphology of the spermatozoa is greater than or equal to 4%:
   if the morphology is below 4%, teratospermia is spoken of.

The vitality of the spermatozoa is normally greater than or equal to 58%.

An abnormal spermogram is normally associated with hypofertility or even infertility.

Thus male infertility is often due to an insufficient number of spermatozoa, to a lack of mobility or a high level of abnormal spermatozoa, these abnormalities being able to be combined, for example in oligoasthenospermic men.

More particularly, it is known (article by Dr Jean-Claude Emperaire, Practitioner's Endocrine Gynecology [French: “Gynecologie endocrinienne du Praticien”], 2007) that a number of spermatozoa below 10 million/ml of ejaculate may in particular be responsible for infertility in men.

The severity of infertility is all the greater when the number of spermatozoa per milliliter does not exceed 5 million (severe oligospermia) and the fertilizing capacity becomes very low and tends towards zero for cases of cryptozoospermia. This fertilizing capacity is however never zero, and any couple may experience authentic pregnancies attributable to the man despite very severe oligospermia or cryptozoospermia.

In practice, it is however not a question of waiting for this rare eventuality, and it is important to attempt to improve the quality of the sperm in order to increase the chances of having natural fertilization and thus avoid having recourse to in vitro fertilization techniques that are very expensive and very stressful and difficult for the patient, which consist of puncturing the testicles or epididymis so as to collect therein the spermatozoa that are situated therein, such as ICSI (Intra Cytoplasmic Sperm Injection) or ISI (Intra Morphologic Sperm Injection), or to a sperm donor, or to adoption.

Numerous studies on the causes of infertility and treatments thereof have already been conducted.

It is for example known, according to the document FR 2964834 that antioxidants such as for example carnitine, zinc, vitamin E, coenzyme Q10 and vitamin B12 or glutathion act on sperm motility. Zinc and vitamin B12 are also described for acting on the production of sperm.

To this end, studies have more particularly been conducted revealing the role of L-carnitine and derivatives thereof, taken alone or in combination with other compounds, on increasing the motility and/or number of spermatozoa in hypofertile men.

The publication by Balercia et al (Placebo-controlled double-blind randomized trial on the use of L-carnitine, L-acetylcarnitine, or combined L-carnitine and L-acetylcarnitine in men with idiopathic asthenozoospermia, 2005) reveals in particular that a treatment of 6 months with 3 g/day of L-carnitine, and more particularly with 3 g/day of L-acetylcarnitine or better still with 2 g/day of L-carnitine in combination with 1 g/day of L-acetylcarnitine, significantly increases the total forward motility of spermatozoa.

Likewise, the publication “Lenzi et al., Use of carnitine therapy in selected cases of male factor infertility: a double-blind crossover trial, 2003” shows the effect of L-carnitine (period of treatment of 2 g/day for 2*2 months preceded by and interspersed with periods of 2 months without treatment) on the increase in the number and total forward motility of spermatozoa in oligoasthenoteratozoospermic patients having in particular a production of spermatozoa of between 10 and 20 million/ml and a total mobility of between 10% and 30% at the time of inclusion.

Other publications tend to confirm these results on the role of L-carnitine taken alone (“Vitali et al., Carnitine supplementation in human idiopathic asthenospermia: Clinical results, 1995”) or in combination with vitamin E (“Wang et al., L-carnitine: safe and effective for asthenozoospermia, 2010”) or with L-acetyl-carnitine (Lenzi et al., A placebo-controlled double-blind randomized trial of the use of combined L-carnitine and L-acetyl-carnitine treatment in men with asthenozoospermia, 2004).

The patent document published under the number EP 2 135 621 A describes a treatment of male infertility by combining two different formulations, which may be administered separately. One of the formulations contains in particular arginine, vitamin A, vitamin E, vitamin B6, vitamin B9, vitamin B12 and coenzyme Q10. The other formulation contains in particular carnitine, arginine, vitamin C and zinc. The experimental studies contained in this document are conducted on infertile couples. They show an increase in the concentration of sperm for patients aimed at in the study, changing from 26,525±25,251 mm³ before treatment to 34,200±28,252 after treatment, that is to say an average increase of around 29%. This document however does not give any indications on the efficacy, or absence of efficacy, of these compositions at different sperm concentrations.

In general, the aforementioned publications neither reveal nor suggest any potentialized effect of L-carnitine, taken alone or in combination with other active principles, on increasing the production of spermatozoa in men having a sperm concentration of below 5 million/ml, which constitutes a specific group of patients, novel and different compared with the groups of patients treated in the prior art cited.

### SUMMARY OF THE INVENTION

Considering the above, one problem that the present invention sets out to solve is using a composition able to improve the production of spermatozoa by the testicles in men producing few spermatozoa, more particularly in men having a sperm concentration below 5 million/ml, that is to say severe oligospermic, cryptozoospermic or azoospermic men. The present invention also proposes to use a composition able to improve the mobility of spermatozoa. It is important, in hypofertile or even infertile men, the spermatozoa produced being nevertheless being able to reach the ejaculate, to enable them to regain natural fertility by substantially increasing the number of fertile spermatozoa produced, and thus to avoid their having to turn to in vitro fertilization, sperm donation or adoption in order to have a child.

Thus the applicant discovered, unexpectedly, that L-carnitine and/or acetyl-L-carnitine or prodrugs thereof taken alone and advantageously in association with a plurality of other active principles acting in synergy has remarkable potentialized activity on the production of spermatozoa when the spermatozoa are only a little produced in men having a sperm concentration of below 5 million/ml, hypofertile or even infertile. The activating effect of L-carnitine, taken alone or in association with a plurality of other active principles acting on the production of spermatozoa is potentialized increasingly respectively in infertile men having fewer than 3 million spermatozoa per milliliter, in infertile men having fewer than 10 million spermatozoa per milliliter and in infertile men having fewer than 1 million spermatozoa per milliliter.

The object of the proposed solution of the invention to this technical problem is a composition comprising L-carnitine and/or acetyl-L-carnitine or prodrugs thereof for use in the treatment of infertility in men having a sperm concentration of less than or equal to 5 million spermatozoa per milliliter.

Advantageously,—the composition comprises docosahexaenoic acid and/or eicosapentaenoic acid;—coenzyme Q10, vitamin E and vitamin B6;—the sperm concentration is less than or equal to 3 million spermatozoa per milliliter, preferably less than or equal to 2 million spermatozoa per milliliter, preferably less than or equal to 1 million spermatozoa per milliliter, preferably less than or equal to 0.5 million spermatozoa per milliliter;—the sperm concentration is less than or equal to 0.1 million spermatozoa per milliliter;—the sperm concentration is greater than 0.1 million spermatozoa per milliliter;—the composition is used in the treatment of infertility in men presenting with asthenospermia or oligoasthenospermia;—the composition further comprises at least one vitamin and/or one micronutrient and at least one trace element;—the vitamin and/or the micronutrient is chosen from vitamin E, vitamin C, vitamin A, vitamin B6, vitamin B8, vitamin B9, vitamin B12, vitamin D, lycopene arginine, N-acetyl-cysteine, coenzyme Q10 and mixtures thereof, preferably vitamin E, vitamin B6 and coenzyme Q10;—the trace element is chosen from zinc, selenium, copper, magnesium, manganese, potassium, iodine and mixtures thereof, preferably zinc and selenium;—the composition comprises coenzyme Q10, vitamin E, vitamin B6, selenium, zinc and docosahexaenoic acid;—the composition comprises a first formulation containing coenzyme Q10, vitamin E, vitamin B6 and optionally omega-3 acid, and a second formulation containing selenium, zinc and L-carnitine and optionally a carbohydrate;—the first formulation is dosed for a daily administration corresponding to 15.0 to 200.0 mg/day of coenzyme Q10, 6.0 to 24.0 mg/day of vitamin E, 0.7 to 2.8 mg/day of vitamin B6 and 600.0 to 1000.0 mg/day of docosahexaenoic acid;—the second formulation is dosed for a daily administration corresponding to 10.0 to 100.0 μg/day of selenium, 5.0 to 20.0 mg/day of zinc, 0.5 to 2.0 g/day of sucrose and 1.0 to 5.0 g/day of L-carnitine;—the first formulation is in the form of a capsule and/or the second formulation is in the form of a sachet to be diluted in a volume of liquid;—the composition is administered orally;—the first and second formulations are administered with a time interval lying between 8 h and 16 h, even more preferentially approximately 12 h, and more advantageously the second formulation is administered in the morning and the first formulation is administered in the evening;—the composition is advantageously administered for a period of at least 3 months; the composition includes fructose, citric acid, selenium, coenzyme Q, vitamin C, zinc, folic acid and vitamin B12;—the composition includes prickly pear seed extract, fish oil, N-acetylcysteine, nopal fruit extract including betalain and quercetin, L-tyrosine, carnitine tartrate, marigold flower extract including lutein, Haematococcus pluvialis extract including astaxanthin, sweet orange fruit extract including hesperidin, green tea leaf extract including polyphenols, Dunaliella salina extract including carotenoids, olive leaf extract including hydroxytyrosol, acerola fruit extract, maritime pine bark extract including oligo-proanthocyanidins, zinc, vitamin B9 and vitamin B12; and—the composition includes zinc, taurine, arginine, vitamin B9, vitamin C, vitamin E, selenium, Coenzyme Q10, fish oil including omega 3.

The invention and the advantages that result therefrom will be understood better from a reading of the following description and non-limitative embodiments.

### DETAILED DESCRIPTION OF THE INVENTION

The composition, preferably oral, according to the invention comprises L-carnitine and/or acetyl-L-carnitine or prodrugs therefore and, advantageously, taken in association with a plurality of other active principles, for treating infertility in men presenting with severe oligospermia, cryptozoospermia or azoospermia.

The composition according to the invention is more particularly intended for hypofertile or even infertile men producing very few spermatozoa, that is to say men producing less than 5 million spermatozoa per milliliter, men producing less than 3 million spermatozoa per milliliter, men producing less than 2 million spermatozoa, in men producing less than 1 million spermatozoa per milliliter or in so-called very severe oligospermic men producing less than 0.5 million spermatozoa per milliliter. The composition according to the invention is also intended for asthenospermic hypofertile men the spermatozoa of whom have mobility of less than 40%.

Oligospermia, severe and very severe oligospermia, cryptozoospermia, azoospermia and/or asthenospermia according to the invention are determined after a thorough examination of the ejaculate of a given patient. The diagnosis is formalized from the results obtained in two successive spermograms produced at an interval of three months.

The composition, preferably oral, according to the invention comprises L-carnitine and/or acetyl-L-carnitine or prodrugs thereof, or the active metabolic derivatives of L-carnitine or acetyl-L-carnitine, taken alone or in combination.

L-carnitine is synthesized from two amino acids: L-lysine and S-adenosyl-methionine in the presence of vitamin C, magnesium, iron and vitamins B3 and B6. It exerts in particular a triple activity: (i) it allows an energetic production from free fatty acids via beta oxidation, the fatty acids binding to coenzyme A in order to form acetyl-CoA and thus being able to cross the internal membrane of the mitochondria by virtue of a specific enzymatic mechanism using L-carnitine as a vehicle; (ii) by eliminating acetyl-CoA, it fulfills a role of antioxidant protecting the DNA and the cell membrane against free radicals; and (iii) it promotes the stability of the mitochrondial membrane by allowing acetylation of the membrane phospholypids. It thus makes it possible, with regard to the spermatozoa, to produce energy for mobility thereof, to combat against oxidative stress and to limit degradation of the mitochondrial membranes.

Unexpectedly, administering L-carnitine particularly greatly increases the number of spermatozoa in men having a sperm concentration of less than 5 million/ml. Likewise, administering L-carnitine promotes mobility of the spermatozoa.

This administration is performed enterally or parenterally. It is a case preferentially of oral administration.

Preferentially, the composition according to the invention further comprises at least vitamin and/or micronutrient and at least one trace element.

By way of non-limitative example, the vitamin and/or micronutrient according to the invention is chosen from vitamin E, vitamin C, vitamin A, vitamin B6, vitamin B8, vitamin B9, vitamin B12, vitamin D, lycopene, arginine, N-acetyl-cysteine and coenzyme Q10, taken alone or in combination.

By way of non-limitative example, the trace element according to the invention is chosen from zinc, selenium, copper, magnesium, manganese, potassium and iodine, taken alone or in combination.

The composition according to the invention may advantageously also comprise extracts of plants preferentially chosen from maritime pine, in particular pycnogenol extracted from maritime pine bark, horse chestnut, polyphenols, maca or dogwood, taken alone or in combination.

Such plant extracts help to improve sperm morphology and concentration and the total number and mobility of spermatozoa, as well as more particularly the concentration and diameter of veins in infertile men with varicocele with regard to horse chestnut.

Preferentially, the composition according to the invention comprises L-carnitine, coenzyme Q10, vitamin E, vitamin B6, selenium, zinc, and also docosahexaenoic acid or eicosapentaeonic acid and a carbohydrate, for example sucrose, as combination product. This plurality of active principles are specifically chosen combined in the composition according to the invention in order to generate and optimize the production and mobility of spermatozoa in oligoasthenospermic men, and in general the quality and more particularly the viability of the spermatozoa thus produced.

This is because the synthesis of the quinone core or coenzyme Q10 from tyrosine is dependent on vitamin B6. The presence of vitamin B6 maximizes the endogenous synthesis of coenzyme Q10. By virtue of this endogenous production, the dose of exogenous coenzyme Q10 administered can be reduced. Furthermore, vitamin E acts as a powerful antioxidant reducing the impact of free radicals at the plasmic membrane and the seminal plasma, while coenzyme Q10 acts at two levels, as antioxidant preventing peroxidation of the plasmic membrane of the spermatozoa so as to limit degradation thereof in the seminal fluid, and as a provider of energy participating in the synthesis of ATP via the Krebs cycle. Supplementation with coenzyme Q10 potentializes the antioxidant action by increasing the plasmatic concentration of tocopherols (vitamin E). This mechanism is dependent on the regenerating capability of coenzyme Q10 on vitamin E. This is because coenzyme Q10 regenerates vitamin E from the tocopheryl radical, after which the vitamin E has fulfilled its antioxidant functions. The prime effect of these three active agents, which act in correlation to limit oxidative stress, is supplemented by the addition of omega-3 fatty acids, more particularly docosahexaenoic acid (DSA) and eicosapentaenoic acid, which form part of the cell membranes and are responsible for maintaining the properties of the lypid bilayer. These membrane lypids of the spermatozoa are important for fluidity, flexibility and mobility and therefore for promoting effective impregnation.

Selenium and zinc supplement the antioxidant action of coenzyme Q10, vitamin E and vitamin B6 by acting more particularly in spermatogenesis and maturation of the spermatozoa thus produced.

Selenium is important for spermatogenesis since it is necessary for biosynthesis of testosterone as well as the formation and normal development of spermatozoa. The role of selenium in spermatogenesis is provided mainly by two selenoproteins: phospholipid hydroperoxide glutathion peroxidase (PHGPx/GPx4) and selenoprotein P. PHGPx/GPx4 has many functions and represents a pivot between selenium, the quality of the sperm and male fertility. Selenoprotein P is a plasmatic protein necessary for the intake of selenium in the testicles. The intake of selenium increases the number of spermatozoa produced by combating oxidative lesions during maturation thereof and improving the sperm quality by reducing the possibilities of structural abnormalities of the flagellum.

Zinc is present in the mitochrondria and in the flagellum of spermatozoa. The intake of zinc prevents deficiencies effecting steroidogenesis (biosynthesis of testosterone). Inducing a small zinc deficiency causes sperm alterations explained by a reduction in the Leydigian function resulting in a reduction in the production of spermatozoa.

Furthermore, sucrose is more particularly used in the composition according to the invention for the role thereof in improving the taste and therefore observance of the taking of the composition according to the invention and in that the hydrolysis thereof into fructose constitutes an energy substrate for the spermatozoa produced.

Preferably, in the context of the present invention, a combination product comprising L-carnitine, coenzyme

Q10, vitamin E, vitamin B6, docosahexaenoic acid, selenium, zinc, and a carbohydrate, for example sucrose, means that said active principles associated can be either present in the same composition or present separately from each other in distinct compositions. In other words, these active principles are intended to be administered in the context of the same treatment, that is to say over a common treatment period, either at the same time, being included or not in a single composition, or at different times. In addition, they are preferably administered by the same oral administration method in identical or different compositions.

Thus the composition according to the invention advantageously comprises one or more formulations, as a combination product for use that is simultaneous, separate or spread over time.

Simultaneous use according to the invention is an administration of the components of the combination product according to the invention included in a single composition or the simultaneous taking of two distinct formulations, for example a capsule comprising a first part of the composition and a sachet diluted in water for the second part of the composition.

Separate use according to the invention is an administration, at the same time, of the compounds of the combination product according to the invention included in at least two distinct compositions, in an identical or different formulation.

Use spread over time according to the invention is a successive administration of the compounds of the combination product according to the invention in at least two distinct compositions, in an identical or different formulation.

The composition according to the invention can advantageously be administered orally, in one or more identical or different formulations. It is in any galenic form normally used for oral administration and in particular in the form of a capsule, tablet, soft capsule, pill, sachet, tube, flask, chewing gum, ball, granule, emulsion, suspension, solution, ampoule, drink, syrup, or powder, preferentially soft capsule and/or sachet.

Advantageously, considering the beneficial correlations between the active principles according to the invention, the composition according to the invention preferentially comprises a first formulation containing coenzyme Q10, vitamin E, vitamin B6 and docosahexaenoic acid, and a second formulation containing selenium, zinc, sucrose and L-carnitine for separate use or use spread over time.

The first formulation is preferentially dosed for a daily administration corresponding to 15.0 to 200.0 mg/day of coenzyme Q10, 6.0 to 24.0 mg/day of vitamin E, 0.7 to 2.8 mg/day of vitamin B6 and 600.0 to 1000.0 mg/day of docosahexaenoic acid, even more preferentially for a daily administration corresponding to 30.0 mg/day of coenzyme Q10, 12.0 mg/day of vitamin E, 1.4 mg/day of vitamin B6 and 800.0 mg/day of docosahexaenoic acid. These preferential ratios have been identified as being suitable for solving the problem according to the invention.

Advantageously, the first formulation is administered in a daily single dose, preferentially before a meal, even more preferentially in the evening before the meal.

Taking this first formulation comprising in particular docosahexaenoic acid preferentially before a meal limits the stagnation of the compounds in the stomach and makes it possible to avoid unpleasant gastric reflux.

In a preferred embodiment of the composition according to the invention, the first formulation is in the form of a soft capsule in particular for administration orally, typically comprising gelatin, a dye and in addition glycerol. The gelatin is for example fish gelatin. The dye is for example an iron oxide.

By way of non-limitative example, the soft capsule advantageously comprises between 70 and 290 mg of fish gelatin for 30 to 135 mg of glycerol and 2.0 to 10.0 mg of iron oxide.

The second formulation is preferentially dosed for daily administration corresponding to 10.0 to 100.0 μg/day of selenium, 5.0 to 20.0 mg/day of zinc, and 1.0 to 5.0 g/day of L-carnitine and optionally 0.5 to 2.0 g/day of saccharose, even more preferentially for a daily administration corresponding to 50.0 μg/day of selenium, 10.0 mg/day of zinc, 1.1 g/day of sucrose and 3.0 g/day of L-carnitine. These preferential ratios have been identified as being suitable for the invention.

Advantageously, the second formulation is administered orally in a daily single dose, preferentially in the morning, even more preferentially in the morning before the meal.

In a preferred embodiment of the composition according to the invention, the second formulation is in the form of a sachet to be diluted in a volume of liquid, in particular water.

By way of example of a method for preparing a composition according to the invention, the two formulations constituting a preferred composition according to the invention are prepared in accordance with the following steps, according to which:

Concerning the manufacture of the first formulation in capsule form:
firstly the content of the capsule is prepared by mixing raw materials in the form of powders comprising coenzyme Q10, vitamin E and vitamin B6 with fish oil containing docosahexaenoic acid in a reservoir, and secondly the casing of the capsule is prepared, comprising fish gelatin, iron oxide and glycerol hot in accordance with a conventional method for forming soft capsules;
the encapsulation of the content in the casing is carried out at a temperature of 20° C. with relative humidity of 25%;
drying is carried out at ambient temperature in order to obtain the first formulation in the form of a capsule; and
each capsule is dosed so as to contain 10.0 mg of coenzyme Q10, 4.0 mg of vitamin E, 0.47 mg of vitamin B6 and 266.67 mg of docosahexaenoic acid.

Concerning the manufacture of the second formulation in the form of a sachet to be diluted:
the various ingredients are weighed separately and the sodium selenite is first of all diluted in part of the sucrose;
all the ingredients (including the L-carnitine and zinc) are next introduced into the mixing vessel, which will perform a first mixing, for example 20 rotations at 8 revolutions/minute;
the mixture is next sieved and calibrated at 1 mm in order to pass into a second vessel that will perform a new mixing, for example 10 rotations at 8 revolutions/minute;
the vessel is next connected to the bagging machine and the mixture transferred into the feed hopper of the bagging machine for bagging, each sachet containing 50.0 μg of selenium, 10.0 mg of zinc, 1.1 g of saccharose and 3.0 g of L-carnitine.

In a preferred embodiment of the composition according to the invention, the administration regime between the first and the second formulation comprises an interval of time of between 8 h and 16 h, even more preferentially approximately 12 h.

Thus, by way of example of a preferred embodiment, in particular in the case where the first formulation of the composition according to the invention is formulated in soft capsules dosed as above and where the second formulation is formulated in sachets to be diluted dosed as above, the indicated dosage is three capsules per day, on one occasion, for example in the evening for the first formulation, and one sachet per day, for example in the morning for the second formulation.

The composition according to the invention is advantageously administered during a period of at least 3 months, corresponding to a period of at least one spermatozoa production cycle, preferentially at least 6 months, that is to say more than two spermatozoa production cycles, or during 1, 2 or even 3 years. Advantageously, such a treatment duration of at least 3 months, and preferentially at least 6 months, will make it possible to attempt to generate the production of sufficient spermatozoa having good mobility and quality by the testicles in men producing few fertile spermatozoa, so as to enable them to regain natural fertility or at least to substantially increase the chances of finding fertile spermatozoa in the ejaculate for in vitro fertilization, and thus to avoid having to turn to sperm donation or adoption in order to have a child.

The composition according to the invention is nutraceutical or pharmaceutical.

Another object of the present invention relates to a food supplement comprising the composition according to the invention.

### EXAMPLE 1

### Compositions COMPRISING CARNITINE ACCORDING to the Invention for Combating Male Infertility

The quantities of ingredients of compositions 1, 2, and 4 described below are the daily quantities administered.

### Composition 1

Composition 1 is in the form of two formulations: in a sachet and in capsules. The daily dose is one sachet in the morning and three capsules in the evening. Three capsules comprise 30.0 mg of coenzyme Q10, 12.0 mg of vitamin E, 1.4 mg of vitamin B6 and 1705 mg of fish oil, including 800.0 mg of docosahexaenoic acid. A sachet comprises 50.0 μg of selenium, 10.0 mg of zinc, 1.1 g of sucrose and 3.0 g of L-carnitine base. The treatment may for example have a duration of 30 days.

### Composition 2

Composition 2 is solely in sachet form. The daily dosage is two sachets per day. Two sachets contain 3.5 g of carnitine fumerate, 1 g of acetyl-L-carnitine, 2 g of fructose, 100 mg of citric acid, 100 μg of selenium, 40 mg of coenzyme Q, 180 mg of vitamin C, 20 mg of zinc, 400 μg of folic acid and 3 μg of vitamin B12. The duration of treatment may for example be 15 days.

### Composition 3

Composition 3 is in the form of two tablets: one non-colored tablet and one colored tablet. The daily dose is four tables per day, including one colored. One colored tablet contains 100 mg of prickly pear seed extract and 100 mg of fish oil, including 2.30 mg of docosahexaenoic acid and 18.20 mg of eicosapentaenoic. One non-colored tablet contains 250 mg of

N-acetylcisteine, 200 mg of nopal fruit extract (including 0.05 mg of betalain and 0.001 mg of quercetin), 150 mg of L-tyrosine, 134.41 mg of carnitine tartrate, 111.12 mg of marigold flower extract (including 5.71 mg of lutein), 85.71 mg of Haematococcus pluvialis extract (including 4.29 mg of astaxanthin), 83.73 mg of sweet orange fruit extract (including 50.24 mg of hesperidin), 67.47 mg of green tea leaf extract (including 20.24 mg of polyphenols), 48 mg of Dunaliella salina extract (including 1.20 mg of carotenoids), 47.19 mg of olive leaf extract (including 0.47 mg of hydroxytyrosol), 40.20 mg of acerola fruit extract (including 30 mg of coenzyme Q10), 20 mg of maritime pine bark extract (including 19 mg of oligo-proanthocyanidins), 15 mg of zinc, 12 mg of vitamin E, 1.40 mg of vitamin B6, 178.49 μg of vitamin B9 and 2.50 μg of vitamin B12. The treatment may last for example for 7 days.

### Composition 4

Composition 4 is in sachet and capsule form. The daily dosage is one sachet and one capsule, preferably in the evening. One sachet contains 15 mg of zinc, 100 mg of taurine, 3 g of carnitine and 100 mg of arginine. One capsule contains 200 μg of vitamin B9, 90 mg of vitamin C, 15 mg of vitamin E, 27.5 mg of selenium, 30 mg of coenzyme Q10, 408 mg of fish oil including 230 mg of omega-3 and 200 mg of docosahexaenoic acid. The treatment may for example extend over 30 days.

### EXAMPLE 2

### Evaluation of the Efficacy of Composition 1 of Example 1 on Male Infertility

The invention is particularly illustrated by the results of the following multicentre observational study carried out in the context (i) of the Reproduction Center of the Pole of Obstetrics, Reproduction and Gynecology (ORG) of the Nice Hospital (Archet II Hospital), (ii) of the Pole of Gynecology-Obstetrics and Reproduction, Biology Department of Reproduction-CECOS of the Bordeaux Hospital (Pellegrin-Maternity Hospital Group) and (iii) of the Marseille Institute of Reproductive Medicine.

The objective of the study was to evaluate the impact of a 6 months treatment with the composition according to the invention as formulated in the above preparation example on the main sperm parameters, namely the sperm number and mobility, in a population of men obtained from three centers distributed in France, consulting for an infertility problem and presenting in particular with oligospermia, cryptospermia, azoospermia, or oligoasthenospermia.

The example focuses on the analysis of the impact of such a treatment with regard to the count in patients having fewer than 3 million spermatozoa per milliliter, and/or the sperm mobility in patients included in the study carried out during at least two spermograms done before the start-of-treatment visit. The need to do two different spermograms in order to determine the pathological character of the values of a spermogram takes account of the personal variability of the results and constitutes a WHO standard.

During the start-of-treatment visit, the composition according to the invention is handed to the patient for treatment of a daily dose for 6 months (1 sachet in the morning and 3 capsules in the evening). The dosages begin the same day.

During the end-of-protocol visit, a post-treatment spermogram is done. Observance is also evaluated.

If the spermogram has at least one non-evaluable result and/or observance has not been complied with, such results are not analyzed.

The sperm count was described and analyzed over 125 patients, including 73 patients presenting with oligospermia, 5 patients presenting with cryptozoospermia, patients presenting with azoospermia, 10 patients presenting with asthenospermia and 33 patients presenting with oligoasthenospermia.

TABLE 1
Results of the sperm count

| | Inclusion visit | Final visit | p value* |
|---|---|---|---|
| Count (M/ml) | 7.35 ± 9.17 | 9.91 ± 13.01 | 0.0088 |
| Av ± SD (Med [IQR]) | (5,00 [1.50; 10.00] | (6.00 [1.00; 12.00] | |

*Student test for matched data

At inclusion, on average the patients had a sperm count of 7.35 M/ml±9.17 as against 9.91 M/ml±13.01 at the final visit, that is to say an average increase of 2.56 M/ml±10.76, corresponding to an average increase of 35%. This difference is statistically significant.

The main analysis was repeated for the purpose of evaluating the change in the number of spermatozoa in patient subgroups: men with a sperm count of less than or equal to one million/ml at inclusion, men with a sperm count of less than or equal to two million/ml at inclusion, men with a sperm count of less than or equal to three million/ml at inclusion, and men with a sperm count greater than three million/ml at inclusion.

The sperm count in men with a sperm count of less than or equal to one million/ml at inclusion was described and analyzed over 29 patients.

TABLE 2
Results of the sperm count in patients who had a count
of less than or equal to one million/ml at inclusion

| | Inclusion visit | Final visit | p value* |
|---|---|---|---|
| Count (M/ml) | 0.20 ± 0.28 | 2.34 ± 4.62 | 0.0171 |
| Av ± SD (Med [IQR]) | (0.10 [0.01; 0.20] | (0.10 [0.01; 2.00] | |

*Student test for matched data

At inclusion, on average the patients had a sperm count of 0.20 M/ml±0.28 as against 2.34 M/ml±4.62 at the final visit, that is to say an average increase of 2.14 M/ml±4.55, corresponding to an average increase of 1070%. This difference is statistically significant.

The sperm count in men with a sperm count of less than or equal to two million/ml at inclusion was described and analyzed over 45 patients.

TABLE 3
Results of the sperm count in patients who had a count
of less than or equal to two million/ml at inclusion

| | Inclusion visit | Final visit | p value* |
|---|---|---|---|
| Count (M/ml) | 0.75 ± 0.80 | 3.22 ± 5.00 | 0.0012 |

*Student test for matched data

At inclusion, on average the patients had a sperm count of 0.75 M/ml±0.80 as against 3.22 M/ml±5.00 at the final visit, that is to say an average increase of 2.47 M/ml±4.77, corresponding to an average increase of 329%. This difference is statistically significant.

The sperm count in men with a sperm count of less than or equal to three million/ml at inclusion were described and analyzed over 51 patients.

TABLE 4
Results of the sperm count in patients who had a count
of less than or equal to three million/ml at inclusion

| | Inclusion visit | Final visit | p value* |
|---|---|---|---|
| Count (M/ml) | 1.00 ± 1.02 | 3.70 ± 5.21 | 0.0002 |

*Student test for matched data

At inclusion, on average the patients had a sperm count of 1.00 M/ml±1.02 as against 3.70 M/ml±5.21 at the final visit, that is to say an average increase of 2.70 M/ml±4.89, corresponding to an average increase of 270%. This difference is statistically significant.

The sperm count in men with a sperm count greater than three million/ml at inclusion were described and analyzed over 74 patients.

TABLE 5
Results of the sperm count in patients who had
a count greater than three million/ml at inclusion

| | Inclusion visit | Final visit | p value* |
|---|---|---|---|
| Count (M/ml) | 11.73 ± 9.72 | 14.19 ± 14.94 | 0.1187 |

*Student test for matched data

At inclusion, on average the patients had a sperm count of 11.73 M/ml±9.72 as against 14.19 M/ml±14.94 at the final visit, that is to say an average increase of 2.47 M/ml±13.43, corresponding to an average increase of 21%. This difference is not statistically significant.

The results demonstrate clearly that the treatment taken daily for six months affords an increase in the production of spermatozoa in infertile patients. More particularly, this increase in the sperm concentration observed following the taking of the treatment is greater in the patients who had a sperm count of less than or equal to three million/ml than in patients who had a sperm count greater than three million/ml. More particularly, the effect on the production of spermatozoa is all the greater in men with a sperm concentration of less than or equal to two million/ml and even more particularly in men with a sperm concentration of less than or equal to one million/ml. This is because the mean increase in the sperm concentration is 1070% in men with a sperm concentration of less than or equal to one million/ml at inclusion, 329% in men with a sperm concentration of less than or equal to two million/ml at inclusion and 270% in men with a sperm concentration of less than or equal to three million/ml at inclusion, as against a mean increase in the number of spermatozoa of 21% in men with a sperm concentration of greater than three million/ml at inclusion.

These results make it possible to identify a subpopulation that is particularly receptive to the treatment. Infertile patients having a sperm concentration of less than or equal to three million/ml therefore represent a subpopulation of patients responding much more effectively to the treatment. The potentialized effect of the treatment on this subpopulation has a surprising and unexpected character.

Finally, the mobility of the spermatozoa was described and analyzed over 28 patients having mobility of less than or equal to 20% at inclusion.

TABLE 6
Results of the sperm mobility in patients who had a
mobility of less than or equal to 20% at inclusion

| | Inclusion visit | Final visit | p value* |
|---|---|---|---|
| Mobility (%) | 9.05 ± 7.22 | 24.61 ± 20.70 | 0.0002 |
| Av ± SD (Med [IQR]) | (9.00 [2.50; 15.00] | (30.00 [1.50; 40.00] | |

*Student test for matched data

At inclusion, on average the patients had a sperm mobility of 9.05%±7.22 against 24.61%±20.70 at the final visit, that is to say an absolute mean increase of 15.55%±19.30, corresponding to a relative mean increase of 172%. This difference is statistically significant.

1. A composition comprising:
at least one selected from the group consisting of L-carnitine, acetyl-L-carnitine, active metabolic derivatives thereof, and combinations of two or more thereof and
at least one selected from the group consisting of docosahexaenoic acid, eicosapentaenoic acid, and combinations of two or more thereof,
wherein the composition is formulated to be effective in the treatment of infertility in men having a sperm concentration less than or equal to 5 million spermatozoa per milliliter.

2. The composition according to claim 1, further comprising:
Coenzyme Q10,
vitamin E, and
vitamin B6.

3. The composition according to claim, 1 wherein the composition is formulated to be effective when the sperm concentration is less than or equal to 3 million spermatozoa per milliliter.

4. The composition according to claim 3, wherein the composition is formulated to be effective when the sperm concentration less than or equal to 0.1 million spermatozoa per milliliter.

5. The composition according to claim 1, wherein the composition is formulated to be effective when the sperm concentration s greater than 0.1 million spermatozoa per milliliter.

6. The composition according to claim 1, wherein the composition is formulated to be effective in the treatment of infertility in men having asthenospermia or oligoasthenospermia.

7. The composition according to claim 1, further comprising:
at least one selected from the group consisting of vitamins, micronutrients, and combinations of two or more thereof, and
at least one trace element.

8. The composition according to claim 7, wherein the one selected from the group consisting of vitamins, micronutrients, and combinations of two or more thereof is selected from the group consisting of vitamin E, vitamin C, vitamin A, vitamin B6, vitamin B8, vitamin B9, vitamin B12, vitamin D, lycopene, arginine, N-acetyl-cysteine, coenzyme Q10, and mixtures of two or more thereof.

9. The composition according to claim 7, wherein the trace element is selected from the group consisting of zinc, selenium, copper, magnesium, manganese, potassium, iodine, and mixtures of two or more thereof.

10. The composition according to claim 1, further comprising:
coenzyme Q10,
vitamin E,
vitamin B6,
selenium,
zinc, and
docosahexaenoic acid.

11. The composition according to claim 10, comprising:
a first formulation containing coenzyme Q10, vitamin E, vitamin B6, and
a second formulation containing selenium, zinc and L-carnitine.

12. The composition according to claim 11, wherein the first formulation is dosed for a daily administration corresponding to 15.0 to 200.0 mg/day of coenzyme Q10, 6.0 to 24.0 mg/day of vitamin E, 0.7 to 2.8 mg/day of vitamin B6 and 600.0 to 1000.0 mg/day of docosahexaenoic acid.

13. The composition according to claim 11, wherein the second formulation is dosed for a daily administration corresponding to an amount in a range of from 10.0 to 100.0 μg/day of selenium, an amount in a range of from 5.0 to 20.0 mg/day of zinc, an amount in range of from 0.5 to 2.0 g/day of sucrose and an amount in a range of from 1.0 to 5.0 g/day of L-carnitine.

14. The composition according to claim 11, wherein at least one selected from the group consisting of:
the first formulation is in the form of a capsule,
the second formulation is in the form of a sachet to be diluted in a volume of liquid.

15. The composition according to claim 1, wherein the composition is formulated to be administered orally.

16. The composition according to claim 11, wherein the first and second formulations are adapted to be administered with a time interval lying between 8 h and 16 h.

17. The composition according to claim 1, wherein the composition is formulated to be effective when administered for a period of at least 3 months.

18. The composition according to claim 1, further comprising:
fructose,
citric acid,
selenium,
Coenzyme Q,
vitamin C,
zinc,
folic acid, and
vitamin B12.

19. The composition according to claim 1, further comprising:
prickly pear seed extract,
fish oil,
N-acetylcysteine,
nopal fruit extract including betalain and quercetin,
L-tyrosine,
carnitine tartrate,
marigold flower extract including lutein,
Haematococcus pluvialis extract including astaxanthin,
sweet orange fruit extract including hesperidin,
green tea leaf extract including polyphenols,
Dunaliella salina extract including carotenoids,
olive leaf extract including hydroxytyrosol,
acerola fruit extract,
maritime pine bark extract including oligo-proanthocyanidins,
zinc,
vitamin B9, and
vitamin B12.

20. The composition according to claim 1, further comprising:
zinc,
taurine,
arginine,
vitamin B9,
vitamin C,
vitamin E,
selenium,
Coenzyme Q10, and
fish oil including omega 3.
